(19) United States
(12) Patent Application Publication
(10) Pub. No.: US 2025/0072524 A1
(43) Pub. Date: Mar. 6, 2025

(54) MEN'S UNDERPANTS ADAPTED FOR RETENTION OF RESIDUAL URINE

(71) Applicant: Jaroslav Kucera, Moravská Nová Ves (CZ)

(72) Inventor: Jaroslav Kucera, Moravská Nová Ves (CZ)

(21) Appl. No.: 18/813,865

(22) Filed: Aug. 23, 2024

(30) Foreign Application Priority Data
Sep. 4, 2023 (CZ) 2023-41261

(51) Int. Cl. A41B 9/02; A41B 9/12; A61F 5/44; A61F 5/453

(52) U.S. Cl. CPC A41B 9/023; A41B 9/12; A61F 5/4401; A61F 5/453

(57) ABSTRACT

The invention relates to men's underpants adapted to capture residual urine, which are provided with a textile layer on the outer surface of the front side in the middle part that does not allow liquid to flow to the outside connected to the outer surface of the front side of the underpants, and between the outer surface of the front side of the underpants and the textile layer is formed a drip space equipped with an absorbent layer. In the central part of the bottoms, below the edge of the bottoms, a narrowing opening reinforced with reinforcement is created, where the opening is equipped with a reinforced longitudinal profile or the size of the opening in its upper part is rigidly or flexibly defined by the upper edge of the bottoms.

### TECHNICAL FIELD

The technical solution relates to men's underpants adapted to catch residual urine.

### STATE OF THE ART

As with women, the problem of incontinence occurs in connection with health issues or advancing age and is currently adequately addressed by the availability of absorbent devices such as incontinence pads or diapers on the market. Absorbent aids for men come in different shapes, sizes and levels of absorption, and underwear with sewn-in absorbent layers for light urine leakage is also available.

However, in addition to incontinence, the male population also faces another problem related not only to health issues and age, but also to the anatomy of the urinary tract, which has a longer urethra and is shaped differently from that of women. If the bladder is not completely emptied, urine accumulates in the urethra leading from the bladder.

There is then a dripping process, whereby this residual urine in the form of the so-called “last drop” spontaneously escapes from the tube by gravity after a certain period of time. There is virtually no prevention or effective solution to this problem. The “last drop” of urine almost always ends up in the underwear. Residual urine can accumulate in the underwear, cause skin staining, leak into the outer clothing or urine can also contaminate the affected person's surroundings and cause hygiene problems and inconvenience to the person and the surroundings.

Addressing this problem with incontinence pads or diapers is not appropriate and does not contribute to psychological comfort. Many people with disabilities do not seek any solution or professional help because of fear and shame.

There is as yet no adequate product on the market for the male population that addresses this delicate problem of the male population without the need for incontinence diapers or other absorbent devices.

### The Essence of the Technical Solution

The above shortcoming is addressed by a technical solution to the modification of men's underpants which directly captures residual urine leaking from the urethra without leaving any traces on underwear, outer clothing or the surrounding area. This modification consists in adjusting the front of the men's underpants so that the urine is trapped at a certain point during normal movement of the urethra after the completion of urination, thus reducing for the most part the splashing of fluid on the garment and the surroundings.

Men's underpants are technically designed to catch urine by being fitted with a textile layer on the outer surface of the front side, in the central part, which does not allow fluid to leak to the outside and which is removably or non-removably bonded to the outer surface of the front side of the underpants. The upper edge of the textile layer is higher than the upper edge of the underpants and is not bonded to the outer surface of the front side of the underpants. A drip compartment is formed between the outer surface of the front side of the underpants and the textile layer, which is provided with an absorbent layer. The drip compartment on the front of the underpants thus forms a barrier against moisture getting onto the outer garment.

The drip compartment is provided with an absorbent layer, which may or may not be removably bonded to a textile layer that does not allow liquid to leak to the outside or may be loosely placed in the drip compartment. The textile layer may be of any shape, for example semi-circular, and may be of any material, and may also absorb the liquid itself.

According to another embodiment of the technical solution, the men's underpants are arranged to catch the urine in such a way that the upper edge of the textile layer is higher than the upper edge of the underpants and the underpants are provided with an opening between the inner part of the underpants and the drip compartment at the front central part, in addition to the fluid-proof layer on the outside. The urethra can be trapped in the opening at the mouth of the absorbent layer after urination is completed and falls over the top edge of the textile layer. The opening may be narrower in the top to bottom direction and the opening is preferably rounded at the bottom. The opening is reinforced at the sides and around the bottom by a reinforcement which is attached at the top of the opening to a retaining edge for the reinforcement. This retaining edge is positioned under the upper edge of the underpants all around their circumference and may be bonded to the upper edge of the underpants at the users hips. The retaining edge for the reinforcement may be, for example, a waistband elastic. The opening may, for example, be reinforced around the perimeter with an underwire instead of a reinforcement.

The men's underpants according to another preferred embodiment are arranged so that the reinforcement is removably or non-removably bonded to a longitudinal reinforced profile open at the top at one end adjacent the bottom of the opening, the other end of the profile being located inside the drip compartment. This profile may be narrowed at the end inside the drip compartment.

The men's underpants according to another preferred embodiment are arranged so that the reinforcement is removably or non-removably bonded to a hollow longitudinal closed reinforced profile at one end adjacent to the bottom of the opening, wherein at the top of this end of the profile, from the edge, a longitudinal rounded cut-out is provided to facilitate the positioning of the urethra within the closed profile. The other end of this profile is located inside the drip compartment and is provided with an end for attaching an attachment, which may be, for example, a tube usable as an extension of the urethra. The vertical cross-section of this profile becomes smaller towards the textile layer. The designs with a reinforced opening and attached reinforced profiles are intended primarily for the capture of urine from a shorter urethra to facilitate its delivery to the absorbent layer.

The men's underpants according to yet another preferred embodiment are arranged such that the upper edge of the textile layer is at the same height as the upper edge of the underpants. According to this embodiment, an opening is formed at the front of the underpants in the central part, wherein the size of the opening is fixedly or flexibly defined at the top by the upper edge of the underpants, for example by sliding elements. The top of the opening of this embodiment may be provided with a sliding element, for example in the form of a tube threaded onto the edge of the underpants to facilitate movement of the urethra into the drip compartment. This design is intended for easy collection of residual urine, especially from the longer urethra.

All designs of men's underpants, according to the technical design, are adapted so that the urethra, after urinating, can easily pass over the edge of the fabric layer into the drip compartment containing an absorbent layer which absorbs the residual urine and the urethra can then return to the underpants by itself, by normal body movement or with gentle assistance.

### CLARIFICATION OF DRAWINGS

FIG. 1: Men's underpants fitted with a semicircular fluid-proof textile layer on the outside forming a drip compartment and an absorbent layer in the drip compartment on the inside of the textile layer

FIG. 2 a) Men's underpants having a textile layer that does not allow liquid to leak to the outside, forming a drip compartment, which is also an absorbent layer, wherein an opening is formed in the front of the underpants from the retaining edge for the reinforcement at the sides and around the bottom part with a reinforcement;

FIG. 2 b) detail of the connection of the reinforcement to the longitudinal reinforced profile open from the top—side view

FIG. 2 c) detail of the connection of the reinforcement to the closed longitudinal reinforced profile with cut-out—side view

FIG. 3: Men's underpants fitted with a textile layer that does not allow liquid to leak to the outside, creating a drip compartment with an absorbent layer, where an opening is created on the front of the underpants, the size of which is flexibly defined at the top by sliding elements located on the upper edge of the underpants.

### EXAMPLES OF IMPLEMENTATION OF THE TECHNICAL SOLUTION

### Example 1

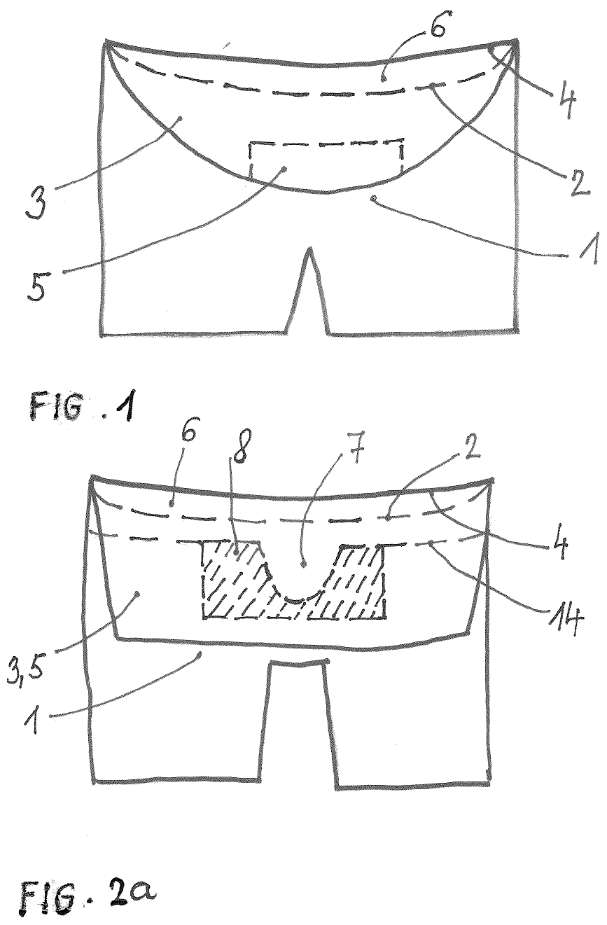

According to the technical solution, the men's underpants are adapted for the collection of urine by being provided with a textile layer 3 on the outer surface 1 of the front side in the central part that does not allow fluid to leak to the outside (FIG. 1). This textile layer 3 is semicircular in shape, is sewn from the sides to the outer surface 1 of the front side of the underpants, its upper edge 4 being higher than the upper edge 2 of the underpants, to which it is not bonded. This textile layer 3 on the front side of the underpants forms a drip compartment 6 on the outer surface of the underpants. The textile layer 3 that does not allow liquid to leak to the outside is provided on the outside with an absorbent layer 5 on the inside in the form of a replaceable thin adhesive liner of absorbent material. Textile layer 3 that does not allow liquid to leak to the outside, forms a barrier against the penetration of moisture that does not reach the outer garment (FIG. 1).

### Example 2

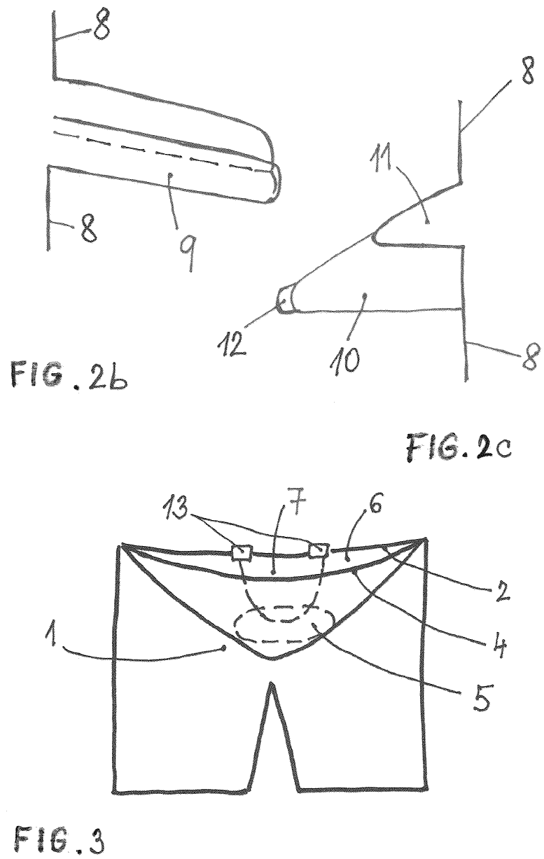

According to the technical solution, the men's underpants are adapted for urine retention by having a cotton textile layer 3 sewn on the outer surface 1 of the front side in the central part, which is also an absorbent layer 5 made by the CityZen® technology (FIG. 2a). This textile layer 3 is rectangular in shape and is attached at the edges by Velcro to the outer surface 1 of the front side of the underpants, its edge 4 being higher than the upper edge 2 of the underpants, to which it is not bonded. This textile layer 3 on the front side of the underpants creates a drip compartment 6. The textile layer 3 that does not allow liquid to leak to the outside forms a barrier against moisture getting onto the outer garment. The underpants are provided with an opening L in the front central part of the underpants in addition to the textile layer 3 that does not allow liquid to leak to the outside. The opening Z narrows from top to bottom and is rounded at the bottom. The opening Z is reinforced at the sides and around the bottom by a reinforcement 8 made of a stronger textile layer than the material of the underpants. Reinforcement 8 is attached at the top of the sides of the opening Z to the retaining edge 14 for the reinforcement 8, which is a waistband elastic sewn horizontally around the entire circumference of the underpants below the upper edge 2 of the underpants in the area of the user's hips.

### Example 3

The men's underpants according to the embodiment of Example 2 for catching residual urine from a shorter urethra are further modified by sewing a hollow longitudinal closed reinforced profile 10 at one end adjacent to the bottom of the opening 7, rounded at its lower part. Profile 10 is made of a thin, flexible plastic/rubber-based material. At the opening Z with reinforcement 8, a longitudinal rounded cut-out 11 is formed on the surface of the profile 10 from its edge in the upper part (FIG. 2c). The other end of this profile 10 is located inside the drip compartment 6 near the absorbent layer § and is provided with an end cap 12 for connecting an extension, which may be, for example, a tube usable as an extension of the urethra. The vertical cross-section of this profile 1Q becomes smaller towards the textile layer 3 that does not allow liquid to leak to the outside. The absorbent layer 5 consists of an absorbent replaceable pulp loosely packed into the drip compartment 6.

### Example 4

Men's underpants according to the technical solution are adapted for the collection of urine by having a textile layer 3 sewn on the outer surface 1 of the front side of the underpants in the central part that does not allow liquid to leak to the outside due to the rubberisation. This textile layer 3 is triangular in shape and is sewn at the edges to the outer surface 1 of the front side of the underpants, its edge 4 being at the same height as the upper edge 2 of the underpants, to which it is not bonded. This textile layer 3 on the front of the underpants creates a drip compartment 6. The textile layer 3 that does not allow liquid to leak to the outside forms a barrier against moisture getting onto the outer garment. The underpants are also provided with an opening Z at the front in the central part, where the size of the opening L is flexibly defined at the top by sliding elements 13 located at the upper edge 2 of the underpants in the form of olivettas. A thin incontinence liner forming an absorbent layer 5 is bonded to the inner side of the textile layer 3. This design is intended for easy collection of residual urine, especially from the longer urethra (FIG. 3).

### INDUSTRIAL APPLICABILITY

The modification of men's underpants provides a discreet solution to an intimate problem in the male population, through a simple, technically and financially inexpensive modification of underwear for everyday use, readily available on the market.

### LIST OF REFERENCE SIGNS

1—outer surface of the front side of the underpants
2—upper edge of the underpants
3—textile layer that does not allow liquid to leak to the outside
4—upper edge of the textile layer
5—absorbent layer
6—drip compartment
7—opening in the front of the underpants
8—reinforcement
9—longitudinal reinforced profile open from the top
10—hollow longitudinal closed reinforced profile
11—cut-out
12—end cap
13—sliding elements
14—retaining edge for reinforcement

1. Men's underpants adapted for the containment of urine, characterized in that they are provided on the outer surface (1) of the front side, in the central part, with a textile layer (3) that does not allow liquid to leak to the outside, bonded to the outer surface (1) of the front side of the underpants, wherein the upper edge (4) of the textile layer (3) is higher than the upper edge (2) of the underpants and is not bonded to the outer surface (1) of the front side of the underpants, wherein a drip compartment (6) is formed between the outer surface (1) of the front side of the underpants and the textile layer (3), which is provided with an absorbent layer (5) removably or non-removably bonded to the textile layer (3) that does not allow liquid to leak to the outside.

2. Men's underpants according to claim 1, characterized in that in the central part of the underpants, below the edge (2) of the underpants, an opening (7) is formed tapering from top to bottom, rounded at the bottom, wherein this opening (7) is reinforced at the sides and around the bottom by a reinforcement (8), which is attached at its upper part on the sides of the opening (7) to a retaining edge (14) for the reinforcement, where the retaining edge (14) is located below the upper edge (2) of the underpants around their circumference.

3. The men's underpants according to claim 2, characterized in that the opening (7) is reinforced along its perimeter by a reinforcement (8), wherein the reinforcement (8) is an underwire.

4. Men's underpants according to claim 2, characterized in that the reinforcement (8) is removably or non-removably bonded to a top open longitudinal reinforced profile (9) at one end adjacent to the bottom of the opening (7), the other end of the profile (9) being located inside the drip compartment (6).

5. Men's underpants according to claim 4, characterized in that the profile (9) is narrowed at the end located inside the drip compartment (6).

6. Men's underpants according to claim 2, characterized in that the reinforcement (8) is removably or non-removably bonded to a hollow longitudinal closed reinforced profile (10) at one end adjacent the bottom of the opening (7), wherein a longitudinal rounded cut-out (11) is formed in the upper part of the profile (10) from the edge of the profile (10), the other end of the profile (10) being located inside the drip compartment (6) and provided with an end cap (12), wherein the vertical cross-section of the profile (10) decreases in size towards the textile layer (3).

7. Men's underpants adapted for catching urine, characterized in that they are provided with a textile layer (3) on the outer surface (1) of the front side in the central part that does not allow liquid to leak to the outside bonded to the outer surface (1) of the front side of the underpants, wherein the upper edge (4) of the textile layer (3) is at the same height as the upper edge (2) of the underpants and is not bonded to the outer surface (1) of the front side of the underpants, wherein a drip compartment (6) is formed between the outer surface (1) of the front side of the underpants and the textile layer (3), which is provided with an absorbent layer (5) removably or non-removably bonded to the textile layer (3) that does not allow liquid to leak to the outside, and an opening (7) is formed in the front side of the underpants in the central part, wherein the size of the opening (7) is fixedly or flexibly defined at its upper part by the upper edge (2) of the underpants.

8. Men's underpants according to claim 7, characterized in that the upper edge (2) of the underpants is provided in the front part with sliding elements (13) flexibly defining the size of the upper part of the opening (7).

9. Men's underpants according to claim 3, characterized in that the reinforcement (8) is removably or non-removably bonded to a top open longitudinal reinforced profile (9) at one end adjacent to the bottom of the opening (7), the other end of the profile (9) being located inside the drip compartment (6).

10. Men's underpants according to claim 3, characterized in that the reinforcement (8) is removably or non-removably bonded to a hollow longitudinal closed reinforced profile (10) at one end adjacent the bottom of the opening (7), wherein a longitudinal rounded cut-out (11) is formed in the upper part of the profile (10) from the edge of the profile (10), the other end of the profile (10) being located inside the drip compartment (6) and provided with an end cap (12), wherein the vertical cross-section of the profile (10) decreases in size towards the textile layer (3).
